(19) United States
(12) Patent Application Publication
(10) Pub. No.: US 2024/0407689 A1
(43) Pub. Date: Dec. 12, 2024

(54) Device for Collecting Biometric and Posture Information for Tracking Consciousness of Rotorcraft Pilot

(71) Applicant: DEFENSE AGENCY FOR TECHNOLOGY AND QUALITY, Jinju-si (KR)

(72) Inventors: Jin Woo AHN, Jinju-si (KR); Seong Hyeon NA, Jinju-si (KR); Jeong Mo KOO, Jinju-si (KR); Wonae SON, Jinju-si (KR)

(21) Appl. No.: 18/605,342

(22) Filed: Mar. 14, 2024

(30) Foreign Application Priority Data
Jun. 9, 2023 (KR) 10-2023-0078257

(51) Int. Cl. A61B 5/18; A61B 5/00; A61B 5/0205; A61B 5/11; A61B 5/16; B64D 43/00; B64D 45/00

(52) U.S. Cl. CPC A61B 5/18; A61B 5/002; A61B 5/0205; A61B 5/163; A61B 5/681; A61B 5/7267; B64D 43/00; B64D 45/00; A61B 5/1116; A61B 5/4803; A61B 2503/22; B64D 2045/0065

(57) ABSTRACT

The present invention relates to a device for collecting a physiological signal, posture information, and the like of a rotorcraft pilot by comprehensively utilizing cameras, on-body wearable devices, control stick pressure detection sensors, and the like while the rotorcraft pilot is in flight to track a state of consciousness of the rotorcraft pilot in real time and transmitting the collected biometric information to an integrated data processing and storage device with a built-in flight data recorder.

### BACKGROUND

### 1. Field of the Invention

The present invention relates to a device for collecting a physiological signal, posture information, and the like of a rotorcraft pilot by comprehensively utilizing cameras, on-body wearable devices, control stick pressure detection sensors, and the like while the rotorcraft pilot is in flight to track a state of consciousness of the rotorcraft pilot in real time and transmitting the collected biometric information to an integrated data processing and storage device with a built-in flight data recorder.

### 2. Discussion of Related Art

Military rotorcraft pilots may always be exposed to stress due to many factors such as battlefield conditions, mission characteristics, and aircraft characteristics (maneuverability characteristics, vibrations, etc.), and may lose consciousness due to high maneuverability, physical characteristics (fainting due to physical and mental tension, etc.), or being shot while performing flight missions. The loss of consciousness of a pilot may lead to aircraft posture instability, an emergency landing, or a crash. Accordingly, a consciousness tracking device based on biometric and posture information is required.

The related art proposes a device and method for tracking the hand temperature, heart rate, and the like of a pilot by utilizing a Hotas-type military aircraft control stick, which is mainly used in fixed wing aircraft, and has a configuration for monitoring the conditions of the pilot by utilizing the hand temperature, heart rate, and the like of the pilot. However, a device for comprehensively collecting body and posture information, such as physiological signals of a rotorcraft pilot, pressure signals applied to collective and cyclic control sticks and pedals, and posture information (including head, shoulder position, etc.) of a pilot, and tracking consciousness based on the collected information has never been implemented.

### SUMMARY OF THE INVENTION

The present invention is for preventing aircraft accidents due to loss of consciousness by constantly tracking the state of consciousness of a rotorcraft pilot through a monitoring device (camera, signal receiving and processing device, etc.), which can check physiological phenomena (pupil responses, blood pressure, pulse, electrocardiogram, oxygen saturation, etc.) of a rotorcraft pilot and posture information (including head, shoulder position, etc.) of the rotorcraft pilot, and transmitting the collected biometric information to a flight data recorder, a mission computer, and the like.

According to an exemplary embodiment, a device for collecting biometric and posture information for tracking a consciousness of a rotorcraft pilot includes: an on-body wearable device worn on a rotorcraft pilot to collect physiological information; a control system pressure signal detection device that detects the pressure applied to a stick and a pedal used to control a rotorcraft: a pilot consciousness and posture tracking and learning device that directly tracks pupil response and posture information of the pilot, receives the physiological information from the on-body wearable device, and derives an anomaly related to a loss of consciousness of the pilot; and an integrated data processing and storage device that is responsible for a condition monitoring function related to the rotorcraft and the pilot and voice and flight data recording, to process and record information received from the pilot consciousness and posture tracking and learning device and the control system pressure signal detection device, and generates a warning signal when identifying an anomaly related to the loss of consciousness.

The rotorcraft may be a military rotorcraft.

The on-body wearable device may include a physiological signal detection module, a Bluetooth transmission/reception module, and a power supply module.

The physiological signal detection module may include a blood pressure detection sensor, a heart rate sensor, an electrocardiogram sensor, a body temperature sensor, and an oxygen saturation sensor, and may be located on a body part including the wrist of the pilot.

The Bluetooth transmission/reception module may transmit the collected physiological information from the physiological signal detection module to the pilot consciousness and posture tracking and learning device.

The power supply module may supply power to the physiological signal detection module and the Bluetooth transmission/reception module, and may be in the form of a built-in battery.

The control system pressure signal detection device may include a pressure detection module, a data transmission/reception module, and a power supply module.

The pressure detection module may include a cyclic stick pressure detection sensor, a collective stick pressure detection sensor, and a pedal pressure detection sensor.

The cyclic stick pressure detection sensor may be located at a lower front center of a cockpit adjacent to the pilot, the collective stick pressure detection sensor may be located in a lower left portion of the cockpit adjacent to the pilot, and the pedal pressure detection sensor may be located in a lower front portion of the cockpit adjacent to the pilot.

The data transmission/reception module may transmit the information collected by the pressure sensing module to the pilot consciousness and posture tracking and learning device, and the integrated data processing and storage device.

The power supply module may supply power to the pressure detection module and the data transmission/reception module.

The pilot consciousness and posture tracking and learning device may include a pupillary response and posture information detection module, a Bluetooth transmission/reception module, a signal information processing and learning module, a data transmission/reception module, and a power supply module.

The pupillary response and posture information detection module may be a camera for confirming a facial part and a posture, and an infrared light for confirming pupillary reflex.

The camera for confirming a facial part and a posture may collect a changing state of the facial part including a pupil, and the posture information including the head, shoulders, and arms of the pilot in real time, and provide the changing state and the posture information to the signal information processing and learning module.

The posture information related to the head, shoulders, and arms of the pilot may be collected in such a way that the camera for confirming a facial part and a posture tracks a virtual vertical line connecting a protrusion of a face including a nose and a chin of the pilot, and a center of a trunk; and a virtual horizontal line across both shoulders.

A movement flow collected during pre-flight inspection of mission equipment or a movement flow collected during operation of a control stick and equipment during flight may be learned through the signal information processing and learning module, and a pilot posture abnormal signal may be transmitted from the signal information processing and learning module when the posture of the pilot deviates from the learning range and takes an abnormal angle.

The infrared light for confirming a pupillary reflex may be a device for confirming a pupillary reflex of a pilot, and may induce a change in pupil size by radiating the infrared light having an infrared band wavelength that is less harmful to a human body toward the pupil.

The change in pupil size of the pilot may be observed in such a way that the camera for confirming a facial part and a posture tracks the pupillary response, and during pre-flight inspection of mission equipment, after confirming an eyeball condition where no pupillary response occurs two or more times, a pupil diameter d before response may be learned through the signal information processing and learning module.

After confirming the pupillary response by radiating the infrared light two or more times during pre-flight inspection of mission equipment or during a flight mission, a pupillary diameter d′ after response determined as an arithmetic mean may be learned through the signal information processing and learning module.

After confirming whether the pupillary response occurs by periodically radiating the infrared light during flight, when d>d′, it may be determined as a state of securing consciousness according to a normal pupillary response, and a signal is transmitted.

When periodically radiating the infrared light during flight and the pupil diameter d′ confirmed through the camera is identified as the pupil diameter d±5% before response, the infrared light may be additionally radiated three or more times at intervals of 1±0.5 seconds to observe a pupillary response, and a pupil diameter d″ obtained by determining the arithmetic mean of the collected pupil diameters may be learned through the signal information processing and learning module, and when d″ is d±5%, it may be determined as a state of loss of consciousness due to no pupillary response, and a signal is transmitted.

The Bluetooth transmission/reception module may receive the physiological information transmitted from the on-body wearable device worn on the pilot and transmit the received physiological information to the signal information processing and learning module.

The signal information processing and learning module may have a function of monitoring the received signal and information, a function of learning and storing the physiological information and posture information of the pilot based on the collected data, and a function of identifying an anomaly related to the loss of consciousness identified from the body information and posture information.

Regarding the physiological information of the pilot, the signal information processing and learning module: may receive information related to a blood pressure, heart rate, electrocardiogram, body temperature, and oxygen saturation from the on-body wearable device at intervals of 5±2 minutes and determine the arithmetic mean of each item to learn an average value in a stable state of consciousness: receive and learn the pupillary diameter d in a stable state of consciousness from the pupillary response and posture information detection module; and when signal information that does not correspond to the learned range is input, determine and process the input as an abnormal input.

Regarding the posture information of the pilot, the signal information processing and learning module: may track movement flows corresponding to positions of the head, shoulders, arms, and hands of the pilot through the camera for confirming a facial part and a posture during pre-flight inspection of mission equipment or during operation of equipment during flight and then learn a normal operating movement flow; and track a manipulation direction and pressure applied to the stick and pedal through the control system pressure signal detection device during pre-flight inspection of mission equipment or during operation of the equipment during flight and then learn the manipulation direction and the pressure range applied at a normal level of consciousness; and when signal information that does not correspond to the learned range is input, determine and process the input as an abnormal input.

Regarding identifying an anomaly related to the loss of consciousness based on the body information and posture information, the signal information processing and learning module: may determine, as a loss of consciousness, when the pupil diameter d″ is d±5% or when the pupil disappears beyond a range of upper and lower eyelids: determine, as a loss of consciousness, when the heart rate measured by the on-body wearable device becomes 0; determine, as a loss of consciousness, when the posture of the pilot is tracked as taking an abnormal angle, and at the same time, when the pressure measured by the control system pressure signal detection device becomes 0; determines, as a loss of consciousness, when the posture of the pilot is tracked as taking an abnormal angle, and at the same time, when the pressure is fixed and applied in a direction corresponding to abnormal operation range in the control system pressure signal detection device; and receives an additional determination criterion related to the loss of consciousness from an outside through the data transmission/reception module.

The data transmission/reception module may transmit the information collected and analyzed in the signal information processing and learning module to the integrated data processing and storage device.

The power supply module may supply power to the pupillary response and posture information detection module, the Bluetooth transmission/reception module, the signal information processing and learning module, and the data transmission/reception module.

The integrated data processing and storage device may include a posture information processing and storage module, a biometric information processing and storage module, a voice and flight record storage device, a data transmission/reception module, and a power supply module.

The posture information processing and storage module may receive and store data related to posture information of the rotorcraft and posture information of the pilot, and generate a warning signal when an anomaly occurs.

The biometric information processing and storage module may receive and store data related to the physiological information of the pilot, and generate a warning signal when an anomaly related to the loss of consciousness of the pilot is identified.

The voice and flight record storage device may be a device that serves as a black box that stores pilot communication data and flight records.

The data transmission/reception module: may receive information from the pilot consciousness and posture tracking and learning device, and the control system pressure signal detection device, and transmit the information to the posture information processing and storage module, the biometric information processing and storage module, and the voice and flight record storage device; and when an anomaly occurs, transmit the signal generated from the posture information processing and storage module or the biometric information processing and storage module to a device that the pilot recognizes using his/her visual and auditory senses, including a multifunction display device.

The power supply module may supply power to the posture information processing and storage module, the biometric information processing and storage module, the voice and flight record storage device, and the data transmission/reception module.

### BRIEF DESCRIPTION OF THE DRAWINGS

FIG. 1 is a configuration diagram of a device for collecting biometric information for tracking the consciousness of a rotorcraft pilot according to the present invention, where P denotes a supply of power, D denotes data transmission and reception according to the MIL-STD-1553B communication protocol, and B denotes data transmission and reception through Bluetooth.

FIG. 2 is a conceptual diagram of the device for collecting biometric information for tracking rotorcraft pilot consciousness according to the present invention.

FIG. 3A is a diagram illustrating the pupillary response tracking of a rotorcraft pilot using a camera for confirming a facial part and a posture and infrared light according to the present invention, particularly illustrating an eyeball state before confirming a pupillary response.

FIG. 3B is a diagram illustrating the pupillary response tracking of a rotorcraft pilot using a camera for confirming a facial part and a posture and infrared light according to the present invention, particularly illustrating a state of clear consciousness (pupil response (reduction), d>d′).

FIG. 3C is a diagram illustrating the pupillary response tracking of a rotorcraft pilot using a camera for confirming a facial part and a posture and infrared light according to the present invention, particularly illustrating a state of loss of consciousness (no pupillary response, d≈d″), where N1 denotes the oculomotor nerve, N2 denotes the optic nerve, d denotes a pupil diameter before testing, and d′ and d″ denote the pupil diameter after testing.

FIG. 4A is a diagram illustrating control stick manipulation posture tracking using the camera for confirming a facial part and a posture according to the present invention, particularly illustrating stick manipulation posture tracking from the front.

FIG. 4B is a diagram illustrating control stick manipulation posture tracking using the camera for confirming a facial part and a posture according to the present invention, particularly illustrating stick manipulation posture tracking from the side.

FIG. 5A is a diagram illustrating head posture and position tracking using the camera for confirming a facial part and a posture according to the present invention, particularly illustrating head posture tracking when normal.

FIG. 5B is a diagram illustrating head posture and position tracking using the camera for confirming a facial part and a posture according to the present invention, particularly illustrating a view when head posture tracking when abnormal.

FIG. 5C is a diagram illustrating head posture and position tracking using the camera for confirming a facial part and a posture according to the present invention, particularly illustrating another view when head posture tracking when abnormal.

FIG. 5D is a diagram illustrating head posture and position tracking using the camera for confirming a facial part and a posture according to the present invention, particularly illustrating a further view when head posture tracking when abnormal.

FIG. 6 is a diagram illustrating the monitoring of physiological information through an on-body wearable device according to the present invention, where I1 denotes blood pressure and heart rate, I2 denotes an electrocardiogram and temperature, and I3 denotes oxygen saturation.

### DETAILED DESCRIPTION OF EXEMPLARY EMBODIMENTS

Hereinafter, the present invention will be described below in detail with reference to the accompanying drawings.

The present invention relates to a device for collecting biometric and posture information for tracking the consciousness of a rotorcraft pilot. The present invention may be preferably applied to military rotorcraft such as a helicopter, but may be applied to civil aircraft as well as other military aircraft.

The loss of consciousness of a pilot may lead to aircraft posture instability, an emergency landing, or a crash. According to the present invention, by monitoring, in real time, biometric information in which a physiological signal and posture information of the pilot are integrated, and storing the collected biometric information, in addition to promoting the safety of on-duty pilots, the biometric information may be used for big data analysis or the like of biorhythm signals for pilot missions in the future.

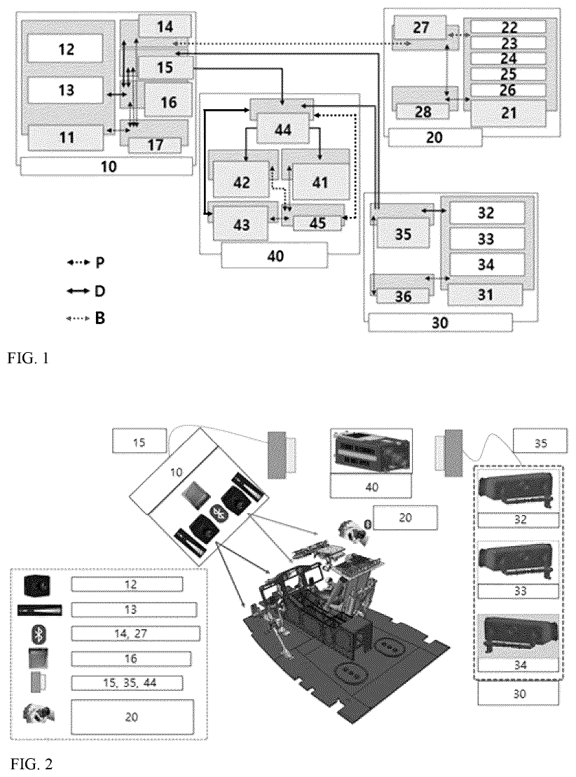

Referring to FIGS. 1 and 2, the device for collecting biometric and posture information for tracking the consciousness of a rotorcraft pilot according to the present invention may be configured of a combination of:
1) a pilot consciousness and posture tracking and learning device 10 that includes a pupillary response and posture information detection module 11 including a camera 12 for confirming a facial part and a posture and infrared light 13 for confirming a pupillary reflex, a Bluetooth transmission/reception module 14, a data transmission/reception module 15, a signal information processing and learning module 16, and the like;
2) a Bluetooth-based on-body wearable device 20 that includes a physiological information confirmation function;
3) a control system pressure signal detection device 30 that includes a control stick (cyclic stick, collective stick, pedal) pressure detection module 31, pressure signal (data) transmission/reception module 35, and the like; and
4) an integrated data processing and storage device 40 that includes a posture information processing and storage module 41, a biometric information processing and storage module 42, a voice and flight record storage device 43, a data transmission/reception module 44, and the like.

The pilot consciousness and posture tracking and learning device 10 may be a sub-device that serves to directly track pupillary response and posture information of the pilot, receive physiological information from the on-body wearable device 20, and derive an anomaly related to the loss of consciousness of a pilot.

The on-body wearable device 20 may be a sub-device worn on a body part such as the wrist of a pilot to collect the physiological information, and transmit data to the pilot consciousness and posture tracking and learning device 10 by a Bluetooth transmission/reception method.

The control system pressure signal detection device 30 may be a sub-device that performs a function of detecting the pressure applied to the cyclic stick, the collective stick, and the pedal used to pilot a rotorcraft, and transmitting the collected information to the pilot consciousness and posture tracking and learning device 10 and the integrated data processing and storage device 40.

The integrated data processing and storage device 40 may be a sub-device that is responsible for a condition monitoring function related to the aircraft and pilot and voice and flight data recording, and serves to process and record information received from the pilot consciousness and posture tracking and learning device 10 and the control system pressure signal detection device 30, and generate a warning signal when identifying an anomaly related to the loss of consciousness.

The pilot consciousness and posture tracking and learning device 10 may include the pupillary response and posture information detection module 11, the Bluetooth transmission/reception module 14, the data transmission/reception module 15, the signal information processing and learning module 16, and a power supply module 17. The pupillary response and posture information detection module 11 may include the camera 12 for confirming a facial part and a posture and the infrared light 13 for confirming a pupillary reflex.

The camera 12 for confirming a facial part and a posture may serve to collect, in real time, posture information, such as a state of change in major parts of the facial part, including a pupil, and positions of the head, shoulders, and arms of a pilot, when operating various instruments for piloting, and transmit the posture information to the signal information processing and learning module 16.

Referring to FIGS. 4A-B and 5A-D, the method of collecting posture information related to the head, shoulders, and arms of a pilot may be performed in such a way that the camera 12 for confirming a facial part and a posture tracks a virtual vertical line connecting a protrusion of a face including a nose and a chin of the pilot, and a center of a trunk; and a virtual horizontal line across both shoulders.

A movement flow collected during pre-flight inspection of mission equipment, or a movement flow during operation of the control stick and equipment during flight may be learned through the signal information processing and learning module 16, and a pilot posture abnormal signal is transmitted from the signal information processing and learning module 16 when the posture of the pilot deviates from the learning range and takes an abnormal angle.

The infrared light 13 for confirming a pupillary reflex is a device for confirming a pupillary reflex of a pilot, and may serve to induce a change in pupil size by radiating the infrared light having an infrared band wavelength that is less harmful to the human body toward the pupil.

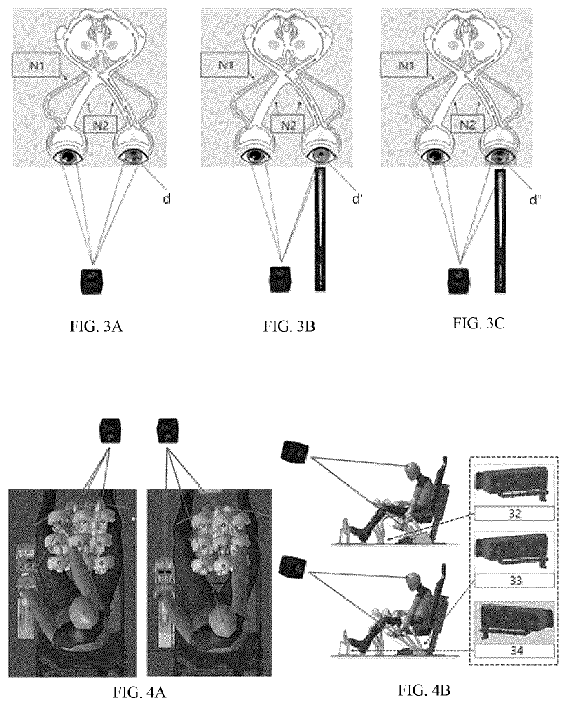

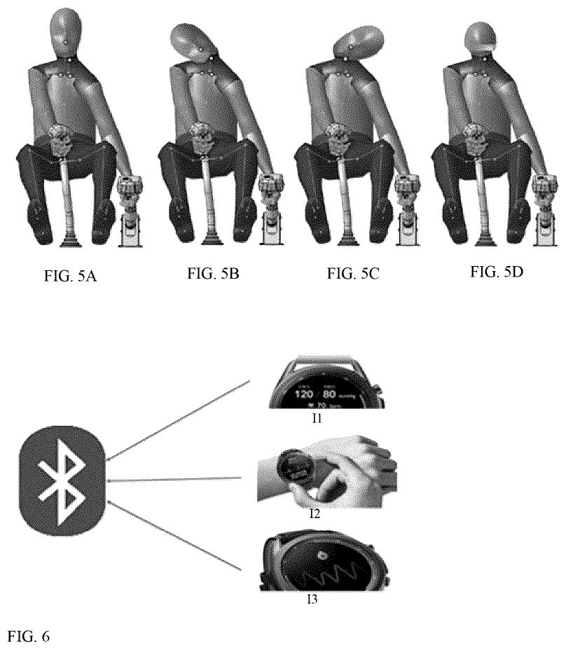

Referring to FIGS. 3A-C, a method of observing a change in pupil size of a pilot may be performed in such a way that the camera 12 for confirming a facial part and a posture tracks a pupillary response.

When inspecting mission equipment before flight, after checking the eyeball state (pupil diameter) where no pupillary response has occurred two or more times, a pupil diameter d before response may be learned through the signal information processing and learning module 16.

Next, after confirming a pupillary response by radiating the infrared light 13 two or more times during pre-flight inspection of mission equipment or during the flight mission, a pupillary diameter d′ after response determined as an arithmetic mean may be learned through the signal information processing and learning module 16.

Next, after confirming whether a pupillary response occurs by periodically radiating infrared light 13 during flight, when d>d′, it may be determined as a state of securing consciousness according to a normal pupillary response, and a signal may be transmitted.

When periodically radiating the infrared light 13 during flight and it is identified that the pupil diameter confirmed through the camera 12 has little difference from the pupil diameter before response, the infrared light 13 may be additionally radiated three or more times at intervals of 1±0.5 seconds to observe the pupillary response, a pupil diameter d″ obtained by determining the arithmetic mean of the collected pupil diameters may be learned through the signal information processing and learning module 16, and when d≈d″ (d″=d±5%), it may be determined as a state of loss of consciousness due to no pupillary response, and a signal may be transmitted.

In this way, the signal information processing and learning module 16 may learn details related to the above-described procedure and transmit a signal.

The Bluetooth transmission/reception module 14 may serve to receive the physiological information transmitted from the on-body wearable device 20 worn on the pilot and transmit the received physiological information to the signal information processing and learning module 16.

The signal information processing and learning module 16 may have a function of monitoring the signal and information received from each module, a function of learning and storing the physiological information and posture characteristics of the pilot based on the collected data, and a function of identifying an anomaly related to the loss of consciousness identified from the body information and posture information. To this end, the signal information processing and learning module 16 may include a processing device (processor), a storage device, and the like. As the learning method, machine learning and deep learning may be used.

Referring to FIGS. 1, 3A-C, and 6, regarding the method of learning and storing the physiological characteristics of a pilot, by transmitting information related to a blood pressure, heart rate, electrocardiogram, body temperature, and oxygen saturation that may be measured by the on-body wearable device 20 to the signal information processing and learning module 16 at intervals of 5±2 minutes, and determining an arithmetic mean of each item, it is possible to learn an average value in a stable state of consciousness.

In addition, as described above, the pupil diameter d in a stable state of consciousness may be transmitted to the signal information processing and learning module 16 and learned. In addition, when signal information that does not correspond to the learned range is input, the signal information processing and learning module 16 may perform processing to determine it as an abnormal input.

Referring to FIGS. 4A-B and 5A-D, regarding the method of learning and storing the posture characteristics of a pilot, the camera 12 for confirming a facial part and a posture may track movement flows corresponding to positions of the head, shoulders, arms, hands, and the like of a pilot during pre-flight inspection of mission equipment or during operation of the equipment during flight, and then transmit the tracked movement flows to the signal information processing and learning module 16 to learn normal operation movement flows.

In addition, the control system pressure signal detection device 30 may track the operation direction and pressure applied to the cyclic stick, the collective stick, and the pedal during pre-flight inspection of mission equipment or during operation of the equipment during flight, and then transmit the tracked operation direction and pressure to the signal information processing and learning module 16 to learn the operation direction and pressure range applied at a normal level of consciousness. In addition, when signal information that does not correspond to the learned range is input, the signal information processing and learning module 16 may perform processing to determine it as an abnormal input.

The signal information processing and learning module 16 may perform the following procedure to identify an anomaly related to the loss of consciousness based on the input body information and posture information.

First, as described above, it may be determined, as a loss of consciousness, when the pupil diameter is d≈d″, or when the pupil disappears beyond the range of upper and lower eyelids.

Second, it may be determined, as a loss of consciousness, when the heart rate measured as described above becomes 0.

Third, it may be determined, as a loss of consciousness, when the posture of the pilot is tracked as taking an abnormal angle as described above, and at the same time, the pressure measured by the control system pressure signal detection device 30 becomes 0 as described above.

Fourth, it may be determined, as a loss of consciousness, when the posture of the pilot is tracked as taking an abnormal angle as described above, and at the same time, when the pressure is fixed and applied in the direction corresponding to the abnormal operating range in the control system pressure signal detection device 30 as described above.

In addition to the previous example, the signal information processing and learning module 16 may receive criteria for additional determination related to the loss of consciousness through the collected body information and posture information from the outside through the data transmission/reception module 15.

The data transmission/reception module 15 may serve to transmit the information collected and analyzed by the signal information processing and learning module 16 to the integrated data processing and storage device 40 according to the MIL-STD-1553B communication protocol.

The power supply module 17 may serve to supply power to the pupillary response and posture information detection module 11, the Bluetooth transmission/reception module 14, the signal information processing and learning module 16, and the data transmission/reception module 15.

Referring to FIG. 6, the on-body wearable device 20 is a device worn on the wrist and the like of a pilot to collect physiological information, and may include a physiological signal detection module 21 that is responsible for a function of detecting and collecting a blood pressure, heart rate, electrocardiogram, body temperature, oxygen saturation, and the like; a Bluetooth transmission/reception module 27 that is responsible for a function of transmitting the collected information to the pilot consciousness and posture tracking and learning device 10; and the like.

Specifically, the on-body wearable device 20 may include the physiological signal detection module 21, the Bluetooth transmission/reception module 27, and a power supply module 28, and may be configured in the form of a watch or a band.

The physiological signal detection module 21 is a device that is located on the wrist and the like of the pilot and is responsible for a function of detecting and collecting a blood pressure, heart rate, electrocardiogram, body temperature, oxygen saturation, and the like, and may be located on a surface that comes in direct contact with the pilot, and may not have a level of accuracy corresponding to a medical device as it is used for tracking a health condition, but provides a detection function corresponding to a level of personal health condition tracking through trend confirmation.

Specifically, the physiological signal detection module 21 may include a blood pressure detection sensor 22, a heart rate sensor 23, an electrocardiogram sensor 24, a body temperature detection sensor 25, an oxygen saturation sensor 26, and the like. These sensors may be intensively located in a watch or band and configured in a modular form.

The Bluetooth transmission/reception module 27 may serve to transmit the information collected from the physiological signal detection module 21 to the pilot consciousness and posture tracking and learning device 10 through Bluetooth communication.

The power supply module 28 may be responsible for supplying power to the physiological signal detection module 21 and the Bluetooth transmission/reception module 27, and may be configured in the form of a built-in battery.

The control system pressure signal detection device 30 may include a pressure detection module 31 that is composed of sensors for detecting and collecting the pressure applied to a cyclic stick, a collective stick, and a pedal; a data transmission/reception module 35 that is responsible for a function of transmitting the collected information to the pilot consciousness and posture tracking and learning device 10 and the integrated data processing and storage device 40; and the like.

Specifically, the control system pressure signal detection device 30 may include a pressure detection module 31, a data transmission/reception module 35, and a power supply module 36.

In order to detect the pressure applied to the control sticks and pedal, the pressure detection module 31 may include a cyclic stick pressure detection sensor 32, a collective stick pressure detection sensor 33, and a pedal pressure detection sensor 34.

The cyclic stick pressure detection sensor 32 may be provided to be located at a lower front center of a cockpit adjacent to a pilot in order to detect an operating pressure of the pilot applied to the cyclic control stick.

The collective stick pressure detection sensor 33 may be provided to be located in a lower left portion of a cockpit adjacent to a pilot in order to detect the operating pressure of the pilot applied to the collective control stick.

The pedal pressure detection sensor 34 may be provided to be located in a lower front portion of a cockpit adjacent to a pilot in order to detect the operating pressure of the pilot applied to the pedal.

The data transmission/reception module 35 may serve to transmit the information collected by the pressure detection module 31 to the pilot consciousness and posture tracking and learning device 10 and the integrated data processing and storage device 40 according to the MIL-STD-1553B communication protocol.

The power supply module 36 may serve to supply power to the pressure detection module 31 and the data transmission/reception module 35.

The integrated data processing and storage device 40 may be responsible for the condition monitoring function related to the aircraft and the pilot, voice and flight data recording, and the like. The integrated data processing and storage device 40 may include a posture information processing and storage module 41, a biometric information processing and storage module 42, a voice and flight record storage device 43, a data transmission/reception module 44, and a power supply module 45.

The posture information processing and storage module 41 may serve to receive and store data related to the posture information of the aircraft and the posture information of the pilot, and generate a warning signal when an anomaly occurs. To this end, the posture information processing and storage module 41 may include a processing device (processor), a storage device, and the like.

The biometric information processing and storage module 42 may serve to receive and store data related to the physiological information of the pilot, and generate a warning signal when an anomaly related to the loss of consciousness of the pilot is identified. To this end, the biometric information processing and storage module 42 may include a processing device (processor), a storage device, and the like.

The voice and flight record storage device 43 is a device that serves as a black box that stores pilot communication data and flight records.

The data transmission/reception module 44 may serve to receive information from the pilot consciousness and posture tracking and learning device 10 and the control system pressure signal detection device 30, and transmit the received information to the posture information processing and storage module 41, the biometric information processing and storage module 42, and the voice and flight record storage device 43. In addition, when an anomaly occurs, the data transmission/reception module 44 may serve to transmit the signal generated from the posture information processing and storage module 41 or the biometric information processing and storage module 42 to a device, such as a multifunction display device, that the pilot may recognize using his/her visual and auditory senses.

The power supply module 45 may serve to supply power to the posture information processing and storage module 41, the biometric information processing and storage module 42, the voice and flight record storage device 43, and the data transmission/reception module 44.

According to a device for collecting biometric information proposed in the present invention, by comprehensively monitoring physiological information (blood pressure, heart rate, electrocardiogram, body temperature, oxygen saturation, etc.) in addition to a pupillary response of a rotorcraft pilot, a head position and steering posture of the rotorcraft pilot, the pressure applied to a control stick, and the like, it is possible to constantly track a state of consciousness of the pilot during flight missions and ground training. In particular, by observing and collecting the body and posture information of the pilot through the device of the present invention even through the pre-flight mission equipment inspection process or simulator training process on the ground, it is possible to identify an anomaly related to the loss of consciousness of the pilot in advance. Based on the above device characteristics, it is possible to promote the safety of on-duty pilots and prevent aircraft accidents due to the loss of consciousness of the pilot. In addition, it is expected that a way to apply the collected biometric information (limited to items with consent to use personal information) to big data analysis, etc., is used for personalized health management for a pilot, biorhythm management of an operational unit level for each aircraft type, and the like.

What is claimed is:

1. A device for collecting biometric and posture information for tracking a consciousness of a rotorcraft pilot, comprising:
an on-body wearable device worn on the rotorcraft pilot to collect physiological information;
a control system pressure signal detection device that detects pressure applied to a stick and a pedal used to control a rotorcraft;
a pilot consciousness and posture tracking and learning device that directly tracks pupil response and posture information of the pilot, receives the physiological information from the on-body wearable device, and derives an anomaly related to a loss of consciousness of the pilot; and
an integrated data processing and storage device that is responsible for a condition monitoring function related to the rotorcraft and the pilot and voice and flight data recording, to process and record information received from the pilot consciousness and posture tracking and learning device and the control system pressure signal detection device, and generate a warning signal when identifying an anomaly related to the loss of consciousness.

2. The device of claim 1, wherein the rotorcraft is a military rotorcraft.

3. The device of claim 1, wherein the on-body wearable device includes a physiological signal detection module, a Bluetooth transmission/reception module, and a power supply module.

4. The device of claim 3, wherein the physiological signal detection module includes a blood pressure detection sensor, a heart rate sensor, an electrocardiogram sensor, a body temperature sensor, and an oxygen saturation sensor, and is located on a body part including a wrist of the pilot.

5. The device of claim 3, wherein the Bluetooth transmission/reception module transmits the collected physiological information from the physiological signal detection module to the pilot consciousness and posture tracking and learning device.

6. The device of claim 3, wherein the power supply module supplies power to the physiological signal detection module and the Bluetooth transmission/reception module, and is in a form of a built-in battery.

7. The device of claim 1, wherein the control system pressure signal detection device includes a pressure detection module, a data transmission/reception module, and a power supply module.

8. The device of claim 7, wherein the pressure detection module includes a cyclic stick pressure detection sensor, a collective stick pressure detection sensor, and a pedal pressure detection sensor.

9. The device of claim 8, wherein the cyclic stick pressure detection sensor is located at a lower front center of a cockpit adjacent to the pilot, the collective stick pressure detection sensor is located in a lower left portion of the cockpit adjacent to the pilot, and the pedal pressure detection sensor is located in a lower front portion of the cockpit adjacent to the pilot.

10. The device of claim 7, wherein the data transmission/reception module transmits the information collected by the pressure sensing module to the pilot consciousness and posture tracking and learning device, and the integrated data processing and storage device.

11. The device of claim 7, wherein the power supply module supplies power to the pressure detection module and the data transmission/reception module.

12. The device method of claim 1, wherein the pilot consciousness and posture tracking and learning device includes a pupillary response and posture information detection module, a Bluetooth transmission/reception module, a signal information processing and learning module, a data transmission/reception module, and a power supply module.

13. The device of claim 12, wherein the pupillary response and posture information detection module includes a camera for confirming a facial part and a posture, and infrared light for confirming a pupillary reflex.

14. The device of claim 13, wherein the camera for confirming a facial part and a posture collects a changing state of the facial part including a pupil, and the posture information including the head, shoulders, and arms of the pilot in real time, and provides the changing state and the posture information to the signal information processing and learning module.

15. The device of claim 14, wherein the posture information related to the head, shoulders, and arms of the pilot is collected in such a way that the camera for confirming a facial part and a posture tracks a virtual vertical line connecting a protrusion of a face including a nose and a chin of the pilot, and a center of a trunk; and a virtual horizontal line across both shoulders.

16. The device of claim 15, wherein a movement flow collected during pre-flight inspection of mission equipment or a movement flow collected during operation of a control stick and equipment during flight is learned through the signal information processing and learning module, and a pilot posture abnormal signal is transmitted from the signal information processing and learning module when the posture of the pilot deviates from the learning range and takes an abnormal angle.

17. The device of claim 13, wherein the infrared light for confirming a pupillary reflex is a device for confirming a pupillary reflex of the pilot, and induces a change in pupil size by radiating the infrared light having an infrared band wavelength that is less harmful to a human body toward the pupil.

18. The device of claim 17, wherein the change in pupil size of the pilot is observed in such a way that the camera for confirming a facial part and a posture tracks the pupillary response, and during pre-flight inspection of mission equipment, after confirming an eyeball condition where no pupillary response occurs two or more times, a pupil diameter d before response is learned through the signal information processing and learning module.

19. The device of claim 18, wherein, after confirming the pupillary response by radiating the infrared light two or more times during pre-flight inspection of mission equipment or during a flight mission, a pupillary diameter d′ after response determined as an arithmetic mean is learned through the signal information processing and learning module.

20. The device of claim 19, wherein, after confirming whether the pupillary response occurs by periodically radiating the infrared light during flight, when d>d′, it is determined as a state of securing consciousness according to a normal pupillary response, and a signal is transmitted.

21. The device of claim 20, wherein, when periodically radiating the infrared light during flight and the pupil diameter d′ confirmed through the camera is identified as the pupil diameter d±5% before response, the infrared light is additionally radiated three or more times at intervals of 1±0.5 seconds to observe a pupillary response, and a pupil diameter d″ obtained by determining the arithmetic mean of the collected pupil diameters is learned through the signal information processing and learning module, and when d″ is d±5%, it is determined as a state of loss of consciousness due to no pupillary response, and a signal is transmitted.

22. The device of claim 12, wherein the Bluetooth transmission/reception module receives the physiological information transmitted from the on-body wearable device worn on the pilot and transmits the received physiological information to the signal information processing and learning module.

23. The device of claim 21, wherein the signal information processing and learning module has a function of monitoring the received signal and information, a function of learning and storing the physiological information and posture information of the pilot based on the collected data, and a function of identifying an anomaly related to the loss of consciousness identified from the body information and posture information.

24. The device of claim 23, wherein, regarding the physiological information of the pilot, the signal information processing and learning module: receives information related to a blood pressure, heart rate, electrocardiogram, body temperature, and oxygen saturation from the on-body wearable device at intervals of 5±2 minutes and determines the arithmetic mean of each item to learn an average value in a stable state of consciousness; receives and learns the pupil diameter d in a stable state of consciousness from the pupillary response and posture information detection module; and when signal information that does not correspond to the learned range is input, determines and processes the input as an abnormal input.

25. The device of claim 23, wherein, regarding the posture information of the pilot, the signal information processing and learning module: tracks movement flows corresponding to positions of the head, shoulders, arms, and hands of the pilot through the camera for confirming a facial part and a posture during pre-flight inspection of mission equipment or during operation of equipment during flight and then learns a normal operating movement flow; tracks a manipulation direction and pressure applied to the stick and pedal through the control system pressure signal detection device during pre-flight inspection of mission equipment or during operation of the equipment during flight and then learns the manipulation direction and the pressure range applied at a normal level of consciousness; and when signal information that does not correspond to the learned range is input, determines and processes the input as an abnormal input.

26. The device of claim 23, wherein, regarding identifying an anomaly related to the loss of consciousness based on the body information and posture information, the signal information processing and learning module: determines, as a loss of consciousness, when the pupil diameter d″ is d±5% or when the pupil disappears beyond a range of upper and lower eyelids; determines, as a loss of consciousness, when the heart rate measured by the on-body wearable device becomes 0; determines, as a loss of consciousness, when the posture of the pilot is tracked as taking an abnormal angle, and at the same time, the pressure measured by the control system pressure signal detection device becomes 0; determines, as a loss of consciousness, when the posture of the pilot is tracked as taking an abnormal angle, and at the same time, when the pressure is fixed and applied in a direction corresponding to abnormal operation range in the control system pressure signal detection device; and receives an additional determination criterion related to the loss of consciousness from an outside through the data transmission/reception module.

27. The device of claim 12, wherein the data transmission/reception module transmits the information collected and analyzed in the signal information processing and learning module to the integrated data processing and storage device.

28. The device of claim 12, wherein the power supply module supplies power to the pupillary response and posture information detection module, the Bluetooth transmission/reception module, the signal information processing and learning module, and the data transmission/reception module.

29. The device of claim 1, wherein the integrated data processing and storage device includes a posture information processing and storage module, a biometric information processing and storage module, a voice and flight record storage device, a data transmission/reception module, and a power supply module.

30. The device of claim 29, wherein the posture information processing and storage module receives and stores data related to posture information of the rotorcraft and posture information of the pilot, and generates a warning signal when an anomaly occurs.

31. The device of claim 29, wherein the biometric information processing and storage module receives and stores data related to the physiological information of the pilot, and generates a warning signal when an anomaly related to the loss of consciousness of the pilot is identified.

32. The device of claim 29, wherein the voice and flight record storage device is a device that serves as a black box that stores pilot communication data and flight records.

33. The device of claim 29, wherein the data transmission/reception module: receives information from the pilot consciousness and posture tracking and learning device, and the control system pressure signal detection device, and transmits the information to the posture information processing and storage module, the biometric information processing and storage module, and the voice and flight record storage device; and when an anomaly occurs, transmits the signal generated from the posture information processing and storage module or the biometric information processing and storage module to a device that the pilot recognizes using his/her visual and auditory senses, including a multifunction display device.

34. The device of claim 29, wherein the power supply module supplies power to the posture information processing and storage module, the biometric information processing and storage module, the voice and flight record storage device, and the data transmission/reception module.
